(19) United States
(12) Patent Application Publication
(10) Pub. No.: US 2016/0213302 A1
(43) Pub. Date: Jul. 28, 2016

(54) SYSTEM AND METHOD FOR SENSING SATIETY AND NOTIFICATION

(71) Applicant: COVIDIEN LP, MANSFIELD, MA (US)

(72) Inventor: SCOTT E.M. FRUSHOUR, BOULDER, CO (US)

(21) Appl. No.: 14/603,453

(22) Filed: Jan. 23, 2015

(51) Int. Cl. A61B 5/00

(52) U.S. Cl. CPC A61B 5/4238; A61B 5/6871; A61B 5/746; A61B 5/7282; A61B 5/0022

(57) ABSTRACT

A gastric sensing system configured to monitor a patient's food intake is provided. The gastric sensing system includes a sensing apparatus adapted to measure satiety of a patient including strain exerted by the stomach. The sensing apparatus includes a sensor, configured to measure satiety of a patient, coupled to a microcontroller and a communication antenna. The communication antenna is configured to wirelessly transmit data collected by the sensors to a remote device. The remote device is in wireless communication with the sensing apparatus and configured to provide information collected by the sensing apparatus to a patient or physician. If the sensing apparatus detects a gastric condition, e.g., stomach strain, exceeds a predetermined threshold, the remote device alerts the patient or physician.

### BACKGROUND

1. Technical Field

The present disclosure relates to systems and methods for sensing satiety of a patient. In particular, the present disclosure provides systems and methods including a sleeve device disposed about a stomach for sensing satiety and communicating sensed conditions to a remote device for monitoring by a patient and/or a clinician.

2. Background of Related Art

Obesity is a leading cause of heart disease and many other serious illnesses, e.g., diabetes, stroke, etc. Recently, there have been a large number of methods and medical devices designed to combat the growing effect of morbid obesity. Some of the treatment methods rely on willpower of the patient, which is usually used in the first instance. When these methods fail, however, surgical intervention is usually required. There are a variety of surgical procedures and devices available to treat obesity including, but not limited to, balloons, bands, rings, and absorption-preventing sleeves. The more invasive of these methods typically include stapling of the stomach, gastric bypass surgery, and the implantation of filler members into the stomach in order to reduce the capacity of the stomach.

Less invasive surgical procedures include positioning of a band about a portion of the stomach so as to reduce or restrict the flow of food through the stomach. These bands can be adjustable or nonadjustable. The adjustable gastric bands allow for adjustment of the restriction of the opening defined by the band so as to reduce or increase the flow of food through the stomach as the patient gains or loses weight. The goal in each of these procedures is to reduce the patient's stomach capacity to restrict the amount of food that the patient can eat. The reduced stomach capacity, in turn, results in a feeling of fullness for the patient after ingesting a relatively smaller amount of food. Thus, the patient can achieve significant weight loss.

However, with many of these procedures and medical devices, a patient must change their lifestyle and eating habits or else risk additional surgical procedures. Thus, there is a need for systems and methods for sensing satiety of a patient and provide feedback regarding the same to the patient and/or doctor in order to ensure the patient is adhering to the prescribed eating regiment.

### SUMMARY

In an aspect, the present disclosure provides a system for sensing satiety of a patient including a sensor assembly and a remote device. The sensor assembly includes a sleeve configured to be disposed about at least a portion of a stomach, at least one sensor disposed on the sleeve and configured to measure at least one property of the stomach and generate a sensor signal, and a microcontroller coupled to the at least one sensor and configured to receive the sensor signal. The remote device is configured to receive a communication signal from the microcontroller based on the sensor signal.

At least one of the sensors of the system may be a strain gauge configured to measure strain exerted by the stomach on the strain gauge. The sensors may be configured to measure tension applied by the stomach. The system may further include a power source. The microcontroller may further be coupled to a communication antenna configured to transmit a communication signal from the microcontroller to the remote device.

The sleeve of the system for sensing satiety of a patient may also be conformable to the stomach and may also include a mesh. The remote device may also be configured to generate an alarm in response to the at least one property exceeding a predetermined threshold.

In another aspect, the instant disclosure provides an apparatus for sensing satiety of a patient including a sleeve configured to be disposed about at least a portion of a stomach. The sleeve includes at least one sensor configured to measure at least one property of the stomach and generate a sensor signal, and a microcontroller coupled to the at least one sensor and configured to receive the sensor signal and transmit the sensor signal to a remote device. At least one of the sensors of the apparatus may be a strain gauge configured to measure strain exerted by the stomach on the strain gauge. At least one of the measured properties of the stomach may be tension applied by the stomach. The apparatus may further include a power source. The microcontroller may also be coupled to a communication antenna configured to transmit a communication signal from the microcontroller to a receiving device. The sleeve may also include a mesh conformable to the stomach.

An aspect of the present disclosure provides for a method for treating obesity including disposing a sleeve about at least a portion of a stomach, the sleeve including at least one sensor configured to measure at least one property of the stomach and generate a sensor signal, measuring at least one property of the stomach; and outputting a measured property on a remote device. The method may further comprise comparing the measured property to a predetermined threshold value. The method may also comprise generating an alarm at the remote device based on the comparison of the measured property to the predetermined threshold value. The at least one sensor may be a strain gauge configured to measure strain exerted by the stomach on the strain gauge. The at least one property may be tension applied by the stomach. The sleeve may also include a mesh conformable to the stomach.

### BRIEF DESCRIPTION OF THE DRAWINGS

Embodiments of the presently disclosed system and method for sensing satiety and notification are disclosed herein with reference to the drawings wherein:

FIG. 1 is an illustration of a system for sensing satiety of a patient and transmitting sensed conditions, according to an embodiment of the present disclosure;

FIG. 2 is a side view of a sleeve of the system of FIG. 1, according to an embodiment of the present disclosure;

FIG. 3 is a schematic block diagram of the sleeve of FIG. 2, according to an embodiment of the present disclosure;

FIG. 4 is a schematic block diagram of a computing device of the system of FIG. 1, according to an embodiment of the present disclosure; and

FIG. 5 is a flow diagram illustrating a method according to an embodiment of the present disclosure.

### DETAILED DESCRIPTION OF THE EMBODIMENTS

Aspects of the present disclosure are described in detail with reference to the drawing figures wherein like reference numerals identify similar or identical elements. As used herein, the term “distal” refers to the portion that is being described which is further from a user, while the term “proximal” refers to the portion that is being described which is closer to a user.

Particular embodiments of the present disclosure are described below with reference to the accompanying drawings; however, it is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to employ the present disclosure in virtually any appropriately detailed structure. In this description, as well as in the drawings, like-referenced numbers represent elements, which may perform the same, similar, or equivalent functions.

Additionally, the present disclosure may be described herein in terms of functional components, optional selections, and various processing steps. It should be appreciated that such functions may be realized by any number of hardware and/or software components configured to perform the specified functions. The present disclosure may employ various integrated circuit components including, but not limited to, memory elements, processing elements, logic elements, look-up tables, and combinations thereof, which may carry out a variety of functions under the control of one or more microprocessors or other control devices.

Further, it should be noted that the present disclosure may employ any number of conventional techniques for data transmission, signaling, data processing, network control, and the like. It should be appreciated that the particular implementations shown and described herein are illustrative of the disclosure and its best mode and are not intended to otherwise limit the scope of the present disclosure in any way.

The scope of the disclosure should be determined by the appended claims and their legal equivalents, rather than by the examples given herein. For example, the steps recited in any method claims may be executed in any order and are not limited to the order presented in the claims.

The present disclosure relates to methods and devices configured to sense satiety of a patient. Embodiments of the present disclosure include a sleeve including sensors configured to measure properties of the stomach. Further, the sleeve includes circuitry and a communication device configured to transmit information collected by the sensors to a remote computing device.

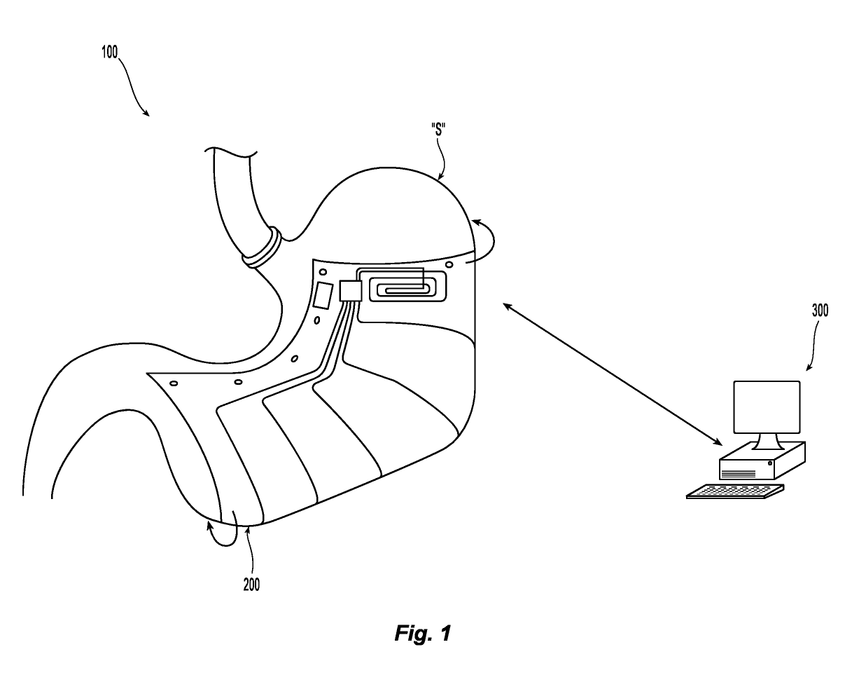

Reference is now made to FIG. 1, which is an illustration of a gastric sensing system 100 in accordance with an embodiment of the invention. Gastric sensing system 100 may include a sensor assembly 200 and a remote device 300, which may be, for example, a personal computer, desktop computer, computer network, mobile phone, or any other suitable computing device. Sensor assembly 200 is configured to be disposed about a stomach “S” and sense one or more gastric conditions, including, but not limited to, satiety of a patient, based on strain and tension exerted by the stomach “S” on sensor assembly 200. Sensor assembly 200 may be surgically disposed about a stomach “S” to help monitor patient activity, such as after a patient undergoes a surgical procedure to treat obesity. Sensor assembly 200 further contains electronic circuitry to wirelessly transmit information collected on satiety of a patient to the remote device 300.

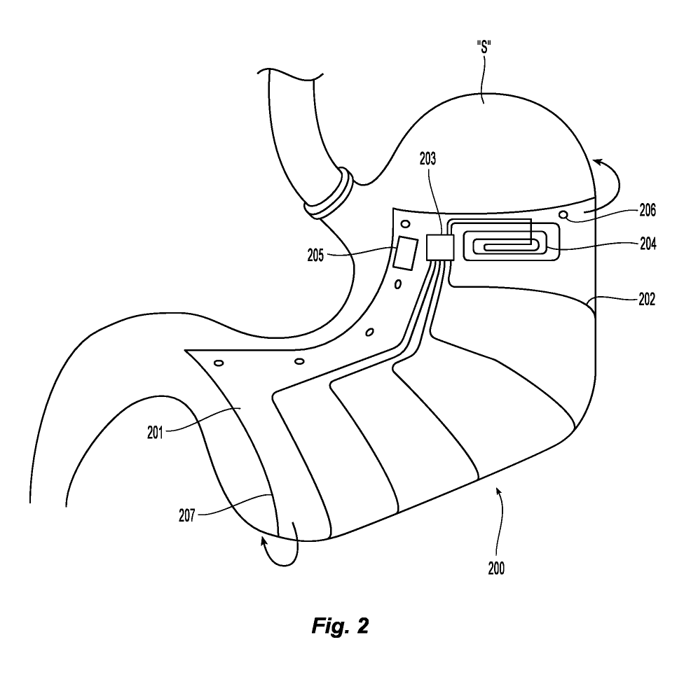

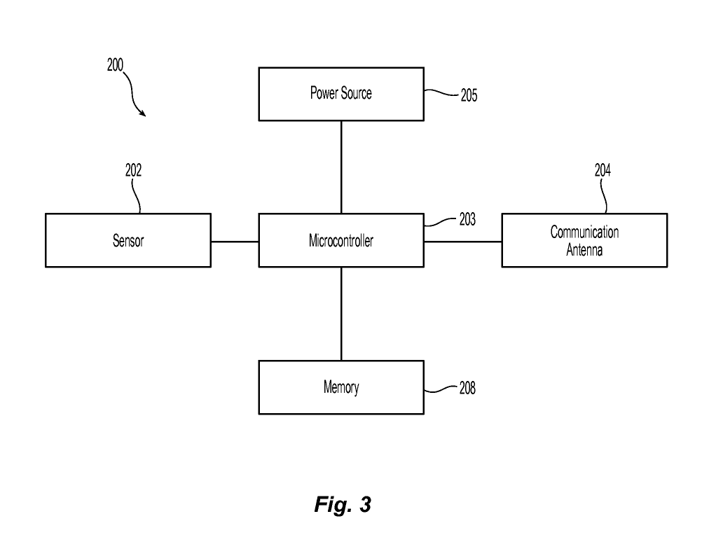

Referring now to FIGS. 2 and 3, sensor assembly 200 may include a sleeve 201, one or more sensors 202, a microcontroller 203, a communication antenna 204, and a power source 205. Sleeve 201 is configured to be disposed around a patient's stomach “S” and may be anchored to the stomach by anchors 206. Anchors 206 may be tabs or staples, or any other suitable anchor capable of securing the sleeve 201 around the stomach “S.” Sleeve 201 is made of any suitable conformable, biocompatible material, including, but not limited to a mesh 207 allowing the sleeve 201 to contour to the shape of the stomach “S.” The mesh 207 may include any suitable biocompatible polymer, such as polypropylene. The mesh 207 material may be woven, non-woven, knitted or braided, and may include a plurality of holes that allow the mesh 207 to be securely attached to the stomach “S.” Although described above and illustrated in FIG. 2 with reference to sleeve 201, other embodiments of sleeve 201 may be contemplated and employed without departing from the scope of the present disclosure. In embodiments, sleeve 201 may be configured as a closed or open loop band that wraps around stomach “S.” Alternatively, sleeve 201 may be an adjustable band.

In some embodiments, sensor assembly 200 may include one or more sensors 202 disposed on the sleeve 201 and configured to sense satiety of a patient, e.g., strain and tension exerted by the stomach “S” on sleeve 201. In one embodiment, the sensors 202 may be strain gauges which measure strain exerted by the stomach “S” as the stomach “S” expands due to food intake. The strain exerted by the stomach “S” as it expands can be correlated to the satiety of a patient. In embodiments, a high strain may indicate a high sense of satiety, and a low strain may indicate a low sense of satiety. Similar sensors configured to output a sensor signal based on impedance in response to varying length due to stretching or contraction of the stomach “S” are also contemplated and may be disposed on the sleeve 201. In embodiments, the sensors 202 may incorporated, e.g., woven, into the sleeve 201.

In some embodiments, sensors 202 may include a combination of more than one type of sensor. Typically, the more sensors used and more types of sensors used, the more accurate sensor assembly 200 may be with measuring strain exerted by the stomach “S.” In other embodiments, sensors 202 may also be configured to sense other gastric conditions in addition to strain, e.g., local/systemic temperature, pH, local/systemic oxygen saturation, bacteria, infections, etc. In some embodiments, sensors 202 are configured to continuously monitor and sense patient satiety, while in other embodiments, sensors 202 are configured to periodically monitor and sense patient satiety.

In some embodiments, each sensor 202 is coupled to a microcontroller 203 which receives signals transmitted by the sensors 202 corresponding to sensed satiety of a patient. Microcontroller 203 may include, or be coupled to, a communication antenna 204 that may transfer signals detected by the sensors 202 to an external remote device, e.g., the remote device 300 as illustrated in FIG. 1. Microcontroller 203 may include, or be coupled to, a power supply 205 for supplying power to all of the electronic components of the sleeve 201.

Power source 205 may be a low voltage rechargeable direct current power supply. The power source 205 may be any suitable power source known to those skilled in the art. In one embodiment, the power source 205 may be rechargeable using an inductive charging interface or other known means of wireless charging.

Microcontroller 203 may further include a microprocessor, logic circuitry, or a semiconductor-based logic device for processing signals generated by at least one of the sensors 202 prior to the signals being transmitted to the remote device 300. The microcontroller 203 may process the signals generated by the sensors 202, such that the transmitted signals may be displayed to a user or may be used to generate a notification to the user with respect to patient satiety. In other embodiments, the microcontroller 203 may perform initial processing of the signals generated by the sensors 202, and then the unprocessed or partially-processed signals may be transmitted to the remote device 300 for further processing.

The strain levels detected by the sensors 202 may be compared to a threshold either prior to being transmitted to the remote device 300, or by the remote device 300. Such analysis of the strain signals detected by the sensors 202 enables not only a determination as to the satiety of the patient but also to a determination as to the extent of satiety, whether mild, progressive or severe, and thus patient treatment may be modified accordingly. In some embodiments, sensors 202 may detect the rate of change of strain, and the rate of change may be compared to a threshold, in order to determine the patient's habits.

In some embodiments, the threshold may either be a predetermined threshold or it may be an adjustable threshold, e.g., a threshold that may change according to the changing levels of strain or may change according to the increase or decrease in the trend of sensed strain levels. An adjustable threshold may, for example, change depending on the time that had passed from surgery. That is, a certain threshold that may be adequate within a short time period post-surgery may no longer be relevant after a longer time period post-surgery.

Microcontroller 203 may further include a memory 208 for storing the signals sensed by the sensors 202. A memory 208 may store a plurality of sensed values collected during operation of the sensor assembly 200 should there be an interruption in communication between microcontroller 203 and the remote device 300. Memory 208 may be an EEPROM (Electronically Erasable Programmable Read-Only Memory) or any other non-transitory storage medium. Communication antenna 204 is configured to wirelessly transmit, or broadcast the detected signals from the sensors 202 (either pre-processing or post-processing) to the remote device 300. The wireless connection may include any of a plurality of communications standards, protocols and technologies, including but not limited to, Global System for Mobile Communications (GSM), Enhanced Data GSM Environment (EDGE), high-speed downlink packet access (HSDPA), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS), and/or Short Message Service (SMS)), or any other suitable communication protocol, including communication protocols not yet developed as of the filing date of this disclosure, and combinations thereof.

According to some embodiments, the remote device 300 may be a mobile device, which may receive the signals related to patient satiety generated by at least one sensor. According to other embodiments, the remote device 300 may include any suitable transceiver for communicating with the sensor assembly 200. In other embodiments, the external remote device 300 may be a wired remote device, i.e., one that microcontroller 203 may be connected to through wires and transmission of data may be done through physical wires. In some embodiments, the remote device 300 may include a processor for processing a signal generated by at least one sensor 202.

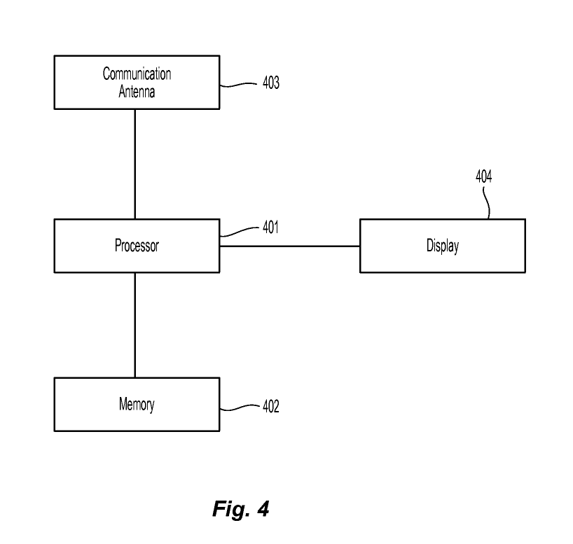

As illustrated in schematic block diagram in FIG. 4, remote device 300 includes various components including, a processor 401, memory 402, a communication antenna 403, and a display unit 404. Remote device 300 may be part of a notification system configured to notify a patient or physician of information pertaining to the patient's satiety. Remote device 300 may be any device that provides output to, and can receive input from, a user, including, but not limited to, a personal computer, laptop, cellular phone, smartphone, etc. In embodiments, output at the remote device 300 may include audio, visual, haptic feedback, or any combination thereof. The remote device 300 may include input components including, but not limited to a keypad, touch screen, mouse, or other device that can accept input, output devices, mass storage media, or other suitable components for receiving, processing, storing, and communicating data.

Communication antenna 403 receives the strain signals detected by the sensors 202 from the communication antenna 204. Communication antenna 403 may also transmit information or data requests to the communication antenna 204 of sensor assembly 200. Remote device 300 may then store the received information in memory 402. The memory 402 allows microcontroller 203 to store a plurality of values collected during operation of the sensor assembly 200. In one embodiment, processor 401 is configured to process the signals generated by the sensors 202 and determine satiety of the patient based on sensor signals from the sensor assembly 200 and determine if the determined satiety of the patient exceeds a predetermined threshold.

Processor 401 may also determine if an indication needs to be generated indicating that a satiety threshold has been reached. The indication may be made by generating an alert for the operator of system 100, e.g., a physician or a nurse or any other healthcare provider, or may be noticed by the patient who underwent the surgical operation. Various types of notifications may be generated by the computing device 300, e.g., an audio alarm (e.g., a high-pitched tone), a visual alarm (e.g., a blinked LED), and/or a contextual alarm displayed on a display unit 404. The display 404 may also output a user interface for managing alert conditions, responding to or forwarding alerts. The user interface may be implemented as a touch user interface.

Display unit 404 may be configured to notify a user of relevant information regarding information on satiety of a patient, including whether or not a threshold value has been reached, and/or current satiety of a patient. In some embodiments, display unit 404 may be a touch screen display and/or be configured to receive input from a user. User input may include a request for current information on satiety of a patient. If a user requests for current information on satiety of a patient, the communication antenna 403 will transmit a request to sensor assembly 200 via the communication antenna 204.

In embodiments, the sensor assembly 200 may communicate with a plurality of computing devices 300 allowing multiple users to be notified of various gastric conditions of a particular patient. Thus, one remote device 300 may be used by a patient and another remote device 300 may be used by a clinician. In further embodiments, the remote device 300 may communicate with multiple sensor assemblies 200 allowing the clinician to monitor conditions of multiple patients.

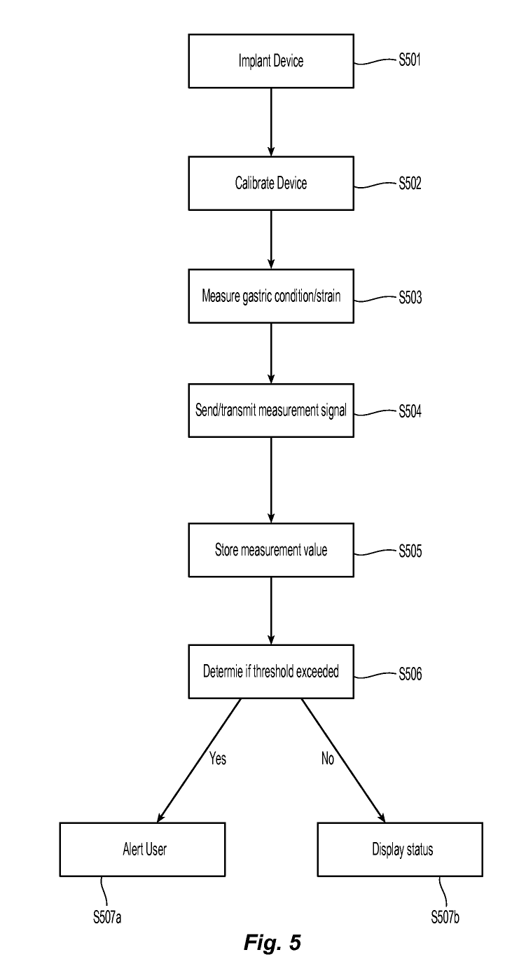

FIG. 5 is a flow diagram illustrating the method of using gastric sensing system 100. The sensor assembly 200 is surgically implanted or installed around the exterior surface of a patient's stomach in S501. The sensor assembly 200 can be anchored in place during surgery with the stomach filled to predetermined amount, e.g., 75% of its total volume. This value can be changed to any given value determined by the surgeon. Given the volume of the stomach during surgery, the sensor assembly 200 may be calibrated in S502 to accurately determine the volume of the stomach at any given time. After surgery, the patient may resume a prescribed eating regimen. The sensor assembly 200 and sensors 202 continually monitor and measure strain or tension exerted by the stomach on the sensor assembly 200 in S503. When the stomach is near empty, the sensors 202 output a low strain value, and when the stomach approaches a certain volume, e.g., 75% of its total volume, the sensors 202 output the calibrated high strain value. In S504, the sensor assembly 200 transmits the value measured by sensors 202 to remote device 300. The measured value is then stored in a memory in S505. The measured value may be stored in either the memory 208 of the sensor assembly 200 or in the memory 402 of the remote device 300. The remote device 300 can then determine whether the measured value has exceeded a predetermined threshold value in S506. In some embodiments, the microcontroller 203 in sensor assembly 200 may determine if the measured value has exceeded a predetermined threshold value. If the measured value has exceeded a predetermined threshold value, remote device 300 alerts a user (e.g., patient and/or clinician) that the patient is abusing the prescribed eating regiment in S507a. If the measured value has not exceeded a predetermined threshold value, the remote device 300 retains the stored value and display a current gastric condition status to the user in S507b.

It will be appreciated that the present invention is not limited to what has been particularly shown and described hereinabove. Rather the scope of the present invention is defined only by the claims which follow.

What is claimed is:

1. A system for sensing satiety of a patient comprising:
a sensor assembly including:
a sleeve configured to be disposed about at least a portion of a stomach;
at least one sensor disposed on the sleeve and configured to measure at least one property of the stomach and generate a sensor signal; and
a microcontroller coupled to the at least one sensor and configured to receive the sensor signal; and
a remote device configured to receive a communication signal from the microcontroller based on the sensor signal.

2. The system of claim 1, wherein the at least one sensor is a strain gauge configured to measure strain exerted by the stomach on the strain gauge.

3. The system of claim 1, wherein the at least one property is tension applied by the stomach.

4. The system of claim 1, wherein the sensor assembly further includes a power source.

5. The system of claim 1, wherein the sensor assembly further includes a communication antenna coupled to the microcontroller, the communication antenna is configured to transmit a communication signal from the microcontroller to the remote device.

6. The system of claim 1, wherein the sleeve is conformable to the stomach.

7. The system of claim 1, wherein the sleeve includes a mesh.

8. The system of claim 1, wherein the remote device is configured to generate an alarm in response to the at least one property exceeding a predetermined threshold.

9. An apparatus for sensing satiety of a patient, which comprises:
a sleeve configured be disposed about at least a portion of a stomach, the sleeve including:
at least one sensor configured to measure at least one property of the stomach and generate a sensor signal; and
a microcontroller coupled to the at least one sensor and configured to receive the sensor signal and transmit the sensor signal to a remote device.

10. The apparatus of claim 9, wherein the at least one sensor is a strain gauge configured to measure strain exerted by the stomach on the strain gauge.

11. The apparatus of claim 9, wherein the sensor assembly further includes a power source.

12. The apparatus of claim 9, wherein the sensor assembly further includes a communication antenna coupled to the microcontroller, the communication antenna is configured to transmit a communication signal from the microcontroller to a receiving device.

13. The apparatus of claim 9, wherein the at least one property is tension applied by the stomach.

14. The apparatus of claim 9, wherein the sleeve includes a mesh conformable to the stomach.

15. A method for treating obesity comprising:
disposing a sleeve about at least a portion of a stomach, the sleeve including at least one sensor configured to measure at least one property of the stomach and generate a sensor signal;
measuring at least one property of the stomach; and
outputting a measured property on a remote device.

16. The method of claim 15, further comprising:
comparing the measured property to a predetermined threshold value.

17. The method of claim 16, further comprising:
generating an alarm at the remote device based on the comparison of the measured property to the predetermined threshold value.

18. The method of claim 15, wherein the at least one sensor is a strain gauge configured to measure strain exerted by the stomach on the strain gauge.

19. The method of claim 15, wherein the at least one property is tension applied by the stomach.

20. The method of claim 15, wherein the sleeve includes a mesh conformable to the stomach.
